(19) United States
(12) Patent Application Publication
(10) Pub. No.: US 2017/0007619 A1
(43) Pub. Date: Jan. 12, 2017

(54) High Dose Dehydroepiandrosterone as Anticancer Treatment With Multi-faceted Reconstitution of Otherwise Depleted Metabolites

(71) Applicant: Jonathan Wesley Nyce, Lansdale, PA (US)

(72) Inventor: Jonathan Wesley Nyce, Lansdale, PA (US)

(21) Appl. No.: 14/791,757

(22) Filed: Jul. 6, 2015

(51) Int. Cl. A61K 31/566; A61K 31/7076; A61K 33/00; A61K 31/375; A61K 31/122; A61K 31/09; A61K 31/4045; A61K 31/675; A61K 31/519; A61K 31/353

(52) U.S. Cl. CPC A61K 31/566; A61K 31/519; A61K 31/7076; A61K 33/00; A61K 31/353; A61K 31/122; A61K 31/09; A61K 31/4045; A61K 31/675; A61K 31/375

(57) ABSTRACT

It has been authoritatively noted that after 100 years of cancer research, the life expectancy in most cancers has only increased by about 3 months. New, effective treatments for cancer are badly needed. The present invention relates to methods to use high doses of Dehydroepiandrosterone, doses sufficient to inhibit tumor Glucose-6-Phosphate Dehydrogenase and thereby deplete tumor NADP(H) pools, with a reconstitution mixture that replenishes metabolites required by normal tissues, thereby avoiding side effects that would otherwise occur. The combination of High Dose Dehydroepiandrosterone and the reconstitution mixture is termed Naturasone™, and its use is described herein both as a replacement for prednisone, and as a standalone cancer medication.

### STATEMENT REGARDING FEDERALLY FUNDED RESEARCH OR DEVELOPMENT

Although the applicant has received several federal research grants in the past (e.g., R01 CA47217; R29 CA47217), the work underlying this invention is unrelated to the work conducted under such grants.

### CROSS REFERENCE TO RELATED PATENTS

None

### SEQUENCE LISTING

Not Applicable

### PRIOR DISCLOSURES

There have been no prior disclosures of this invention

### BACKGROUND OF THE INVENTION

1. Field of the Invention

The field is directed to compositions and methods related to a novel treatment of cancer in which high doses of Dehydroepiandrosterone (DHEA, CAS 53-43-0), doses sufficiently high to inhibit tumor Glucose-6-phosphate dehydrogenase (G6PD, CAS 9001-40-5) and thereby deplete tumor Nicotinamide Adenine Dinucleotide Phosphate, reduced form (NADPH, CAS 53-59-8), are supplemented with specific metabolites in order to prevent certain negative consequences of such high dose DHEA, such metabolites including tetrahydrobiotperin (BH4, CAS 17528-72-2); N6-Δ2-isopentenyladenosine (IPA, CAS 7724-76-7); certain precursors and cofactors of monoamine biosynthesis; folinic acid (CAS 58-05-9); ubiquinone (CAS 303-98-0) and tocotrienols; and a nitric oxide donor such as potassium nitrate (7757-79-1).

2. Description of Related Art

Approximately 1.7 million Americans are diagnosed with cancer each year, and approximately 600,000 will die each year from cancer (National Cancer Institute, Annual Cancer Statistics, United States, http://seer.cancer.gov/statfacts/). Worldwide, about 14 million new cases of cancer are diagnosed annually, and more than 8 million die from cancer (World Cancer Research Fund International, http://www.wcrf.org/int/cancer-facts-figures/worldwide-data). It has been authoritatively stated that more than 100 years of research into cancer treatment has resulted in an increased life span of as little as 3 months for many, if not most forms of cancer. (See Siddhartha Mukherjee, The Emperor of All Maladies, a Biography of Cancer; Simon & Schuster, 2010). In dogs, the situation is even worse due to their increased cancer risk as compared to humans. (See Torres de la Riva, G et al, PLoS One, 2013, 8(2):e55937). There thus remains a critical need to augment current treatment modalities, and to devise new, better treatment modalities for both human and veterinary cancer.

Normal cells undergo a process of differentiation in which they mature into the adult cell type required for their integrated participation in the tissue of which they are a part. The end stage of this process of differentiation is apoptosis, or programmed cell death. The cancer cell possesses faults in its capacity to differentiate, does not integrate into the tissue of which it is a part but instead exceeds its natural boundaries, metastasizing to distant sites, and develops resistance to both apoptosis (programmed cell death) and immune surveillance. There are both genetic (mutation driven) and epigenetic (without mutation) mechanisms of cancer initiation and development underlying their inability to differentiate and undergo normal programmed cell death and identification and removal by the host immune system.

Most classical chemotherapy agents, almost all of which are still in use today, target rapidly dividing cells. Although cancer cells do frequently have cell cycle times that are shortened compared to their nonmalignant counterparts, there are few cancer cells which have rates of cell division that surpass those of rapidly replicating normal cells (e.g., epithelial tissues such as those that line the gastrointestinal tract, hair follicles, etc.). This fact underlies the frequent side effects of cytotoxic chemotherapy drugs. Most cancer cells have shortened cell cycle times (i.e., increased rates of DNA synthesis) because they have mutations that disable certain cell cycle checkpoints. An example is the G1 checkpoint at which normal cells assess their DNA for the presence of lesions that could be fixed into the DNA as permanent mutations if they are not repaired prior to DNA synthesis. Because mutations in specific genes (e.g., the p53 tumor suppressor gene) destroy the G1 checkpoint (See, for example, Prudhomme, M, Recent Pat Anticancer Drug Discov 1(1):55-68, 2006), malignant cells enter DNA synthesis with their DNA damage unrepaired, thus acquiring more and more mutations with every cell division. When these mutations occur in protein-coding regions or regions of DNA that control the transcription of proteins, enormous heterogeneity is introduced into the tumor cell population. Such heterogeneity creates a huge pool of physically altered proteins and altered expression patterns of normal proteins from which drug resistant variants (clones) may be selected for during future attempts at treatment.

For example, resistance to cytosine arabinoside (araC; CAS 147-94-4), a drug commonly used to treat leukemia, can occur by natural selection favoring those few cells in a leukemic cell population that do not express active deoxycytidine kinase (either by epigenetic gene silencing or mutation). Deoxycytidine kinase (dCK, CAS 9039-45-6) is a protein that is required for activation of araC to its active form. Alternatively, resistance to araC can occur via amplification of the cytidine deaminase (CD, CAS 9025-05-2) gene. CD is a protein which deactivates araC to its non-toxic uracil derivative. In yet another variation on this theme, resistance to araC may occur in a leukemia cell population via mutation in the site at which the drug binds to dCK, or at the site in dCK where ATP, the phosphate donor in this enzymatic reaction, binds. In either case, the ability of leukemic clones possessing such modifications decreases their ability to activate araC. Even small changes in araC activation may be sufficient to confer a selective advantage to leukemia cell clones possessing them, creating a drug resistant subpopulation within the cancer cell population—toxicity will occur in normal cells before it does in such a drug-resistant population. Such mechanisms of drug resistance occur for virtually all small molecule drugs in current use. A new drug which reduced mutation rate in cancer cell populations, and which maintained the expression of critical activating genes by preventing their epigenetic silencing, would represent a significant improvement over currently available cancer drugs.

There are several non-surgical approaches to cancer treatment, including cytotoxic chemotherapy, as with araC, noted above; the use of cancer-selective antibodies; the induction of a tumor-specific immune response by injection into the tumor of polio or other viruses or bacteria; differentiation therapy; and stimulation of the host immune system. Differentiation therapy is designed not to kill the cancer cell outright (as in cytotoxic chemotherapy) but rather to induce the cell to undergo a more substantive differentiation than it has heretofore been capable of. The goal is to induce either apoptosis, or the silencing of expression of genes that confer a protective advantage to the cancer cell. Among this latter category falls a cell surface protein called CD47. CD47 has been described as a “don't eat me” signal to the immune system. It is present on the surface of all normal cells when they are young, and its expression gradually decreases until during the end stages of differentiation, when the cell is approaching apoptosis, expression reaches zero and the cell becomes visible to the immune system. Because almost all cancer cells fail to differentiate, they fail to extinguish the expression of CD47, and remain invisible to the immune system. One goal of some cancer differentiation agents, such as the agent of the present invention (see below), is to induce apoptosis and/or the extinction of CD47 gene expression. Agents capable of inducing differentiation to the extent of inducing either apoptosis or extinction of CD47 expression would represent an important addition to the clinical protocols currently available to treat human and veterinary cancer.

Another way that cancer cells escape immune surveillance is by secretion of extremely high amounts of adenosine into the tumor microenvironment. Adenosine potently inactivates a variety of immune system effector cells by activating their adenosine A2A receptors. Although methods to prevent adenosine secretion by tumors and subsequent inactivation of immune cells by activation of their A2A receptors are under discussion, none are yet in use in man or animals. The present invention describes a method to achieve adenosine depletion in the tumor microenvironment, thereby deactivating A2A receptors and activating host immune effector cells.

Still other pathways play important roles in most tumors. Mutations of two genes, one coding for the Ras oncoprotein and the other coding for the p53 tumor suppressor protein, have been found to occur in a high percentage of all animal and human cancers. The Ras oncoprotein requires isoprenylation (enzymatic addition of hydrophobic isoprene units) before it can attach to the inner surface of the plasma membrane, the only site from which it can exert its tumorigenic effects. The p53 tumor suppressor protein has many functions, one of which is to inhibit the enzyme glucose-6-phosphate dehydrogenase (G6PD), the rate limiting step of the pentose phosphate pathway (PPP). Up regulation of the PPP in cancer cells appears to account for much or all of the classic observation that cancer cells preferentially synthesize ATP via glycolysis, instead of by oxidative phosphorylation in the mitochondria. Accordingly, a novel drug treatment that could inhibit the isoprenylation of the Ras oncoprotein, thereby inactivating it, and assume one or more roles of missing p53 tumor suppressor function in cancer cells, e.g., the inhibition of G6PD, would represent a significant improvement in cancer chemotherapy.

An example of a current, widely used cytotoxic chemotherapy protocol is CHOP. CHOP stands for the combination of the following drugs: Cytoxan (cyclophosphamide), Hydroxydaunomycin (doxorubicin), Oncovin (vinblastine) and Prednisone. The CHOP protocol is primarily used to treat lymphoma, both human and veterinary. While CHOP is capable of inducing remission in some cases of lymphoma, cure with this treatment is virtually nonexistent. The lympholytic activity of prednisone produces some short term benefit to both human and veterinary patients with lymphoma, but its longer term use is accompanied by a host of side effects including loss of activity due to induction of drug metabolizing enzymes, the indiscriminate destruction of lymphocytes (damaging host immune response to the cancer), and the development of Cushing's disease. If substitutes for prednisone could be found that were more effective than prednisone and had fewer side effects, this would represent an important innovation in cancer chemotherapy that could immediately improve current protocols.

There are several known forms of drug resistance to the CHOP protocol. Over expression of the gene for glucose-6-phosphate dehydrogenase (G6PD) is associated with the development of resistance to cyclophosphamide (Tsukamoto N, et. al., Blood Cells Mol Dis. 1998, 24(2):231-8), and doxorubicin (Polimeni, M et. al., Biochem. J. 2011, 439, 141-149). Certain p53 tumor suppressor gene mutations also create a drug resistant phenotype for doxorubicin. (See Aas, T, et. al., Nat Med. 1996 July; 2(7):811-4; Chan, K T and Lung, M L, Cancer Chemother Pharmacol. 2004 June; 53(6):519-26. Epub 2004 Mar. 4). Clearly, a novel agent that could not only replace prednisone and improve upon prednisone's anticancer activity, but also block drug resistance to some of the other drugs in the CHOP protocol, would represent an important improvement in cancer treatment. If this agent also showed activity as differentiation monotherapy against solid tumors for which there are currently no effective treatments and if it reduced a tumor's ability to evade the immune system, its importance would be still further enhanced.

Dehydroepiandrosterone (DHEA; CAS 53-43-0) is a natural steroid hormone that has been proposed as a potential cancer chemopreventative. (For a review see Williams, J, Lipids, 04/2000; 35(3):325-31). It has never before been successfully used to treat cancer. It is known that DHEA is an uncompetitive inhibitor of G6PD, the rate limiting enzyme of the hexose monophosphate shunt, and a major source of cellular NADPH. NADPH plays major roles as a reducing agent for many cellular biochemical reactions. In addition to inhibiting G6PD, DHEA also serves as a precursor for the synthesis of steroid hormones. Certain analogs of DHEA cannot be metabolized to steroid hormones include 7-fluoro-dehydroepiandrosterone (fluasterone) and 7-bromo-dehydroepiandrosterone.

DHEA/DHEAS has been proposed as a treatment or prevention in humans for a variety of diseases including vaginal atrophy, hypogonadism, diminished libido, osteoporosis, urinary incontinence, ovarian cancer, uterine cancer, skin atrophy, for contraception, and, in combination with an estradiol and/or progestin, for the treatment of menopause (Labrie, F., European Patent EP0680327); depression (Michael, Herbert, WIPO Patent Application WO/1996/025164); Systemic Lupus Erythematosus (McGuire, James L., U.S. Pat. No. 5,567,696); and Primary Adrenal Deficiency and Addison's Disease (Yen, Samuel S. C. and Berger, Brian, WIPO Patent Application WO/1998/032445). The author of the instant patent has several issued patents on the use of DHEA in the treatment of cancer, asthma and other diseases. (Nyce, Jonathan W., U.S. Pat. No. 7,893,044; Nyce, Jonathan W., U.S. Pat. No. 5,527,789; European Patent EP0627921; Nyce, Jonathan W., U.S. Pat. No. 6,087,351; Nyce, Jonathan W., U.S. Pat. No. 6,670,349; Nyce, Jonathan W., U.S. Pat. No. 7,456,161).

The use of DHEA to prevent or treat chronic fatigue syndrome and/or fibromyalgia in humans, dogs and cats has been proposed (Zenk, Ronald, Zenk, John L., WIPO Patent Application WO/2002/043737), but this invention specifically excluded doses of DHEA that could lead to the synthesis of steroid hormones. Also, the treatment or prevention of male or female menopause symptoms using a combination of a sex hormone binding globulin synthesis inhibiting agent and one or more steroids has been reported (Van Der, Hoop Roland Gerritsen, WIPO Patent Application WO/2003/002123), but this application focuses on improvement of libido and/or sexual response.

The use of high dose DHEA sufficient to inhibit G6PD in the treatment of cancer, supplemented with specific metabolites to prevent side effects associated with depletion of NADP(H), has not heretofore been proposed.

### BRIEF SUMMARY OF THE INVENTION

Our novel data indicate that when doses sufficiently high to deplete tumor NADPH levels are utilized, positive effects in tumor inhibition, life extension, and improvements in quality of life occur. Our novel data further demonstrate, however, that such high doses of DHEA induce potentially catastrophic auto inflammatory disease resulting from depletion of tetrahydrobiopterin (BH4), and N6-Δ2-isopentenyladenosine (IPA). Additional negative effects occur with high dose DHEA, including loss of nitric oxide control of vasodilation; depletion of critical monoamines such as serotonin, dopamine, epinephrine and melatonin; and increased levels of phenylalanine, creating a condition resembling phenylketonuria.

Our novel data indicate that such auto inflammatory disease, loss of control of vasodilation, depletion of monoamines, and increased levels of phenylalanine induced by high dose DHEA treatment can be prevented by co-administration of specific metabolites, including BH4, IPA, a nitric oxide donor such as potassium nitrate, monoamine precursors and cofactors, folinic acid, ubiquinone and tocotrienols. This, for the first time, permits high dose DHEA to be administered at doses sufficient to induce sustained tumor NADPH depletion in vivo. The combination of high dose (HD) DHEA, BH4, IPA, nitric oxide donor, monoamine precursors and cofactors, ubiquinone and tocotrienols, with or without folinic acid rescue, is alternatively referred to as Naturasone (Naturasone™).

When Naturasone replaces prednisone in the CHOP protocol, it produces a similar lympholytic effect as prednisone by inhibition of NADP(H)-dependent pathways including the folate and mevalonate pathways. Because of its inhibition of the folate pathway, the source of cellular purines, Naturasone also depletes the ability of the tumor to secrete adenosine into its microenvironment, thereby eliminating its ability to disarm immune effector cells by activation of the adenosine A2A receptors. Even when employed as monotherapy in place of the CHOP protocol, Naturasone can produce dramatic decreases in lymphadenopathy.

Naturasone's utility as a replacement for prednisone has additional effects not observed with prednisone. Thus, because DHEA inhibits G6PD, when it replaces prednisone in the CHOP protocol it also blocks one of the major mechanisms of drug resistance to both cyclophosphamide and doxorubicin—the up-regulation of G6PD in cancer cells. In fact, G6PD is upregulated in most, if not all cancer cells as part of the program of malignancy. Whereas prednisone does not inhibit G6PD, the DHEA in Naturasone is a potent uncompetitive inhibitor of G6PD. Thus, used as monotherapy or combination chemotherapy in cancers expressing high levels of G6PD, Naturasone possesses multiple important benefits over prednisone.

The present invention therefore describes methods to achieve positive outcomes in cancer with Naturasone, used either alone or in combination with other chemotherapy drugs, such positive outcomes measured as (1) tumor inhibition, (2) life extension, and/or (3) improvements in quality of life. The substance and novelty of this invention rests in our discovery that, at doses of DHEA high enough to induce DHEA depletion and anticancer activity, the above noted metabolites must be added to the formulation to prevent potentially catastrophic DHEA-induced auto inflammatory disease and other negative effects. This novel combination of agents permits Naturasone to be used as a replacement for prednisone in various multi-drug regimens, and as monotherapy in differentiation-sensitive and/or G6PD-expressing cancers, both human and veterinary.

In order to fully illustrate the utility of this invention, the metabolites that become depleted during high dose DHEA treatment must be discussed.

IPA. Our laboratory has made the novel finding that IPA, a product of the mevalonate pathway, becomes depleted when exogenous DHEA is administered in amounts sufficient to induce HD DHEA. IPA plays several important roles in the cell, and its depletion would be expected to be deleterious on many levels. For example, in the biosynthesis of selenoproteins, the adenosine at residue 37 of tRNA molecules that bind codons starting with UGA (normally a stop signal in mRNA) is modified to create IPA within the tRNA molecule (Bifulco, M. Malfitano, A M, Proto, M C et al. Anticancer Agents Med Chem 2008, 8(2): 2000-2004). The T4 deiodinase that converts inactive T4 to active T3 is such a selenoprotein. There are at least 24 additional mammalian selenoproteins, the identification and investigation of which has been hampered by the fact that the selenocysteine insertion signal is UGA, which heretofore had been considered exclusively a stop codon; hence most selenoproteins were unidentified in genetic databases until newer algorithms were devised to identify their presence. (Kryukov, G. V. et al, J. Biol. Chem. 274, 33888-33897, 1999). IPA depletion not only impairs the synthesis/function of the selenoprotein T4 deiodinase, but may also exert an oncogenic pressure upon the cell, Thus, Spinola et al have shown that the enzyme (TRIT1) that catalyzes the transfer of the isopentenyl moiety to the target tRNA is 6-14 fold down-regulated in lung adenocarcinomas as compared to normal lung tissue (Spinola M et al, Oncogene. 2005 Aug. 18; 24(35):5502-9). This identifies TRIT1 as a tumor suppressor gene. IPA is then identified as a tumor suppressor molecule whose absence in DHEA-treated animals would almost certainly be oncogenic, as it would cause the same diminution of IPA addition to tRNA that TRIT1 down-regulation causes. This shows that, in animals treated with DHEA to induce HD DHEA, it is critical to replenish IPA to maintain the tumor suppressor activity of TRIT1. The tumor suppressor function of selenoproteins was also demonstrated in an animal model (Hudson, T. S. et al, Carcinogenesis 33(6):1225-1230, 2012). Indeed, pharmacological doses of DHEA have been shown to sometimes cause cancer rather than prevent it (See Hayashi F., Carcinogenesis. 1994 October; 15(10):2215-9). This can now be explained as due to the DHEA-mediated depletion of IPA, with the downstream depletion of selenomethane, leading to the same endpoint—depletion of selenoproteins—as would occur with TRIT1 tumor suppressor gene inactivation.

Selenoproteins, and therefore IPA, have also been shown to play a critical role in inflammation. For example, loss of function of the selenoprotein Sep15 leads to dramatic induction of STAT-1 regulated inflammatory genes. (See Tsuji, P. A. et al, PLoS One, 2015, 10(4):e0124487). Selenoprotein synthesis is a critical determinant of the balanced biosynthesis of pro- and anti-inflammatory oxylipids in macrophages. (See Mattmiller, S A et al, J. Nut Biochem 25(6):647-54, 2014). Auto-inflammatory conditions caused by genetic defects in the mevalonate pathway (in which the isopentenyl moiety is synthesized) have been described (e.g., Caso, Francesco et al., Int J. Rheumatol 2013, Oct. 24; van der Burgh, R et al, Clin Immunol 147(3):197-206, 2013). Aspects of these monogenic auto-inflammatory diseases closely resemble some of the symptoms observed in animals treated with DHEA to induce HD DHEA. These include fever, inflammation of the eyes, skin and serous membranes.

IPA also regulates Natural Killer (NK) cell activity. NK cells are lymphocytes of the innate immune system that can kill transformed and pathogen-infected cells directly. They also secrete a variety of cytokines and chemokines (e.g., TNF-α, IFN-γ, CCL3, CCL5, IL-8, IL-10, etc) through which they shape the subsequent adaptive immune response. Properly functioning NK cells therefore play critical roles in immune defense and regulation of inflammatory responses. IPA induces expansion and activation of the NK cell compartment. DHEA has been shown to increase NK cell number and cytotoxicity (Khorram, O et al, J Gerontol A Biol Sci Med Sci. 1997 January; 52(1):M1-7). During HD DHEA, when DHEA stimulates NK cell activity and NK-mediated cytotoxicity, it is at the same time eliminating IPA and therefore the controls upon NK cell activity and cytotoxicity that IPA provides. NK cells are known to moderate the inflammatory response in the eye. (Liu Q et al, Am J Pathol. 2012 August; 181(2):452-62). We see identical pathology in dogs treated with DHEA to restore steroid hormone levels to normal via HD DHEA. Evidence that such ocular pathology is caused by IPA depletion comes from the fact that reconstitution of IPA (and BH4) prevents such DHEA-induced ocular inflammation from occurring.

Since we have observed that motor incoordination sometimes occurs in animals undergoing HD DHEA, it is important to note that reduction in neuronal selenoprotein synthesis has been demonstrated to lead to loss of motor coordination in mice. (See Seeher, S. et al, Biochem J. 462(1):67-75, 2014).

It had been thought that the sole source of IPA was the turnover of isopentenylated tRNA. However, studies in yeast (Laten, H M and Zahareas-Doktor, S PNAS USA February 1985; 82(4):1113-1115) and plants (Stot, Crister A, Karel Dolezal, Anders Nordstrom et al. PNAS USA Dec. 19, 2000; 97(26):14778-14783) show that the major source of free IPA is not turnover of isopentenylated tRNA, but rather a separate pathway independent of such turnover. Similar means to synthesize free IPA must also exist in animal tissues, as is supported by several findings. First, statins (anti-cholesterol drugs that inhibit HMG CoA reductase, the rate limiting step in isoprenoid and cholesterol synthesis) inhibit cell growth and division by arresting cells in the G1 phase of the cell cycle. Isopentenyladenine (the free base form of IPA) is approximately 100 fold more active in restoring DNA synthesis in statin-treated cells than is mevalonate, the direct product of HMG Co A reductase (See Huneeus, V Q, Wiley, M H and Siperstein, M D, PNAS USA October 1980: 77(10):5842-5846). Second, we have discovered that addition of IPA can completely prevent the ophthalmic inflammatory reaction that can occur in dogs following administration of DHEA (see below). These findings, along with the studies in yeast and plants noted above, suggest that all organisms possess synthetic pathways to produce free IPA, and that such IPA has important growth regulatory and/or immunoregulatory roles. An alternative way to replenish IPA, rather than administration of IPA itself, is to administer mevalonate, the precursor molecule for all isoprenoid moieties, or geranylgeraniol, geraniol, or farnesol, which are natural isoprenoid donors.

BH4. Our laboratory has also made the novel finding that the pteridine BH4 is depleted in animals undergoing HD DHEA. BH4 is a required cofactor for many critical enzyme systems including four aromatic amino acid hydroxylases, alkylglycerol mono-oxygenase and three NOS (NO synthase) isoenzymes. It thus plays a critical role in monoamine neurotransmitter formation, cardiovascular and endothelial function, the immune response and pain sensitivity. Within the brain, BH4 is absolutely required for the synthesis of the monoamine (MA) neurotransmitters dopamine (DA), norepinephrine, epinephrine (E), and serotonin (5-HT), the novel gaseous neurotransmitter nitric oxide and the production of yet to be fully identified 1-O-alkylglycerol-derived lipids. (See Kapatos, G., The neurobiology of tetrahydrobiopterin biosynthesis, IUBMB Life 65(4):323-33, 2103). Depression (Pan, L et al, Brit Med J Case Rep bcr0320113927, 2011) and hypopigmentation of the skin have also been reported in animals and persons with BH4 deficiency. (Nagasakin, Y. et al., Pediatr Res. 1999 April; 45(4 Pt 1):465-73).

DHEA-mediated BH4 depletion during induction of HD DHEA can lead to increased levels of tissue phenylalanine, creating a situation mimicking the genetic disease phenylketonuria (PKN). PKN is known to cause symptoms similar to those we have observed in dogs and cats administered DHEA to induce HD DHEA, including eczema/atopic dermatitis, an increased incidence of pyogenic infections, an increased incidence of keratosis pilaris, scleroderma-like plaques, and hair loss. (See Scriver, C R and Clow, C L, Annu Rev Genet. 1980; 14:179-202). In human PKU caused by mutations in one of several genes involved in BH4 synthesis, including GCH1, PCBD1, PTS, and QDPR, leading to BH4 deficiency, symptoms can be largely eliminated by pharmacological treatment with BH4 or a BH4 precursor molecule (Sanford, Mark; Keating, Gillian M., 2009, “Sapropterin”. Drugs 69 (4): 461-76). Other methods to reduce phenylalanine levels consequent to BH4 deficiency and prevent the negative sequellae of excessive phenylalanine include (1) dietary restriction so as not to ingest foods high in phenylalanine (van Spronsen F J¹, Enns G M, Mol Genet Metab. 2010; 99 Suppl 1:S90-5. doi: 10.1016/j.ymgme.2009.10.008), (2) dietary use of casein glycomacropeptide (a milk-derived peptide containing extremely low amounts of phenylalanine (Strisciuglio P and Concolino D, Metabolites. 2014 Nov. 4; 4(4):1007-17. doi: 10.3390/metabo4041007;), and (3) dietary supplementation with large neutral amino acids (LNAAs, e.g. leucine, tyrosine, tryptophan, methionine, histidine, isoleucine, valine, threonine, etc.) which compete with phenylalanine for specific carrier proteins that transport LNAAs across the intestinal mucosa into the blood and across the blood brain barrier into the brain (Ney D M, Blank R D, and Hansen K E, Curr Opin Clin Nutr Metab Care. 2014 January; 17(1):61-8). Each of these techniques is envisioned as additional embodiments of the instant invention to reduce phenylalanine levels in animals depleted of BH4 during HD DHEA, although reconstitution of BH4 by supplementation with BH4 or its prodrug, Sepiaopterin, remains the preferred embodiment for DHEA-mediated BH4 depletion (see below).

Nitric Oxide (NO). BH4 is a necessary cofactor for the enzymatic synthesis of NO, as noted above, and NO is depleted during HD DHEA. However, BH4 reconstitution is insufficient to reconstitute NOS activity because NADPH is also required for NO synthesis, and NADPH is diminished during HD DHEA. However, various NO donors are available that can reconstitute NO non-enzymatically. For example, potassium nitrate (KNO₃, CAS 7757-79-1), a common food preservative, directly breaks down to NO after ingestion, and can replenish NO under clinical conditions in which it has been depleted (See Baliga, R S, et al, Respiratory and Critical Care Medicine, Abstract Issue, B63, Experimental models in pulmonary hypertension, American Thoracic Society International Conference Abstracts, 2012). We have made the novel observation that potassium nitrate can replenish NO in animals undergoing HD DHEA, eliminating the symptoms associated with NO depletion.

NO is important as a toxic defense molecule against infectious organisms. It also regulates the functional activity, growth and death of many immune and inflammatory cell types including macrophages, T lymphocytes, antigen-presenting cells, mast cells, neutrophils and natural killer cells.(See Coleman, J W, Int Immunopharmacol. 2001 August; 1(8):1397-406). Increased susceptibility to infections occurs when NO is limiting (See Olekhnovitch, R et al, J. Clin Invest 124(4):1711-1722, 2014). As noted above, susceptibility to infections, particularly in the ear and eye, have been noted in dogs and cats treated with DHEA in amounts sufficient to induce HD DHEA. Again, such susceptibility to infections is eliminated when DHEA treatment includes concurrent BH4 (potassium nitrate and IPA; see below).

Some vascular effects, primarily an induction of vasoconstriction, have been observed during HD DHEA, and these appear to be entirely due to NO depletion since they can be prevented by co-administration of NO donors as noted above. NO was originally described as endothelial relaxing factor, and when it becomes depleted, blood vessels become constricted, increasing blood pressure, decreasing blood flow, and leading to fluid retention in limbs (Sharma, R and Davidoff, M N, Oxidative stress and endothelial dysfunction in heart failure, Congest Heart Fail. 2002 May-June; 8(3):165-72). Fluid retention subsequent to NO depletion has been observed in dogs treated with DHEA in amounts sufficient to induce HD DHEA. Such symptoms are eliminated when treatment includes concurrent BH4, potassium nitrate and IPA.

Ubiquinone depletion. Ubiquinone (CAS Number 606-06-4; Coenzyme Q10) is a critical component of the mitochondrial respiratory chain, participating in electron transport in NADH-coenzyme Q reductase (complex I), succinate coenzyme Q reductase (complex II) and the cytochrome system. (See Nakamaru-Ogiso E et. al., J Bioenerg Biomembr. 2014 August; 46(4):269-77). It is especially important with respect to the function of muscle tissue in organs with high energy expenditure, for example in the heart. (Khorrami A, et. al., Drug Res (Stuttg). 2014 April; 64(4):177-81). Over the short term, ubiquinone levels are increased upon DHEA exposure. However, after continuous treatment with DHEA at doses capable of inducing HD DHEA, ubiquinone levels decline. (FIG. 3). We have found that supplementation with ubiquinone and tocotrienols maintains normal or near normal ubiquinone levels in animals treated with DHEA to induce HD DHEA (see below). Supplementation with the combination of ubiquinone and tocotrienols appears to be more effective at retaining normal or near normal ubiquinone levels than does supplementation with ubiquinone alone. (See Bentinger, M, et. al. Biofactors. 2008; 32 (1-4):99-111) have recently reported that tocotrienols stimulate the synthesis of endogenous ubiquinone, and it is possible that this effect is responsible for the dramatically improved reconstitution when ubiquinone plus tocotrienols were used in our experiments.

Depletion of folate pathway intermediates. HD DHEA depresses NADPH production via its inhibition of G6PD. Therefore, pathways that depend heavily on NADPH are inhibited during HD DHEA. The one carbon pool (folate) pathway is one such pathway. We have discovered that one carbon pool products (e.g., purines, pyrimidines, S-adenosylmethionine) are profoundly depleted during HD DHEA. This is similar to the effect of methotrexate (CAS 59-05-2), a cancer chemotherapy agent which inhibits the rate limiting enzyme of the one carbon pool pathway, Dihydrofolate Reductase (CAS CAS9002-03-3; See Schalinske1, K L and Steele, R D Carcinogenesis vol. 17 no. 8 pp. 1695-1700, 1996). Methotrexate is often administered to cancer patients at doses high enough to be lethal to the patient, but with folinic acid “rescue” to reconstitute one carbon pool metabolism in a timely enough fashion to prevent such lethality (See Borsi, J D et al, Pediatr Hematol Oncol. 1990; 7(4):347-63; folinic acid CAS 1492-18-8). One carbon pool disruption during HD DHEA can have very positive effects with respect to reduction of the adenosine levels in tumor microenvironments, adenosine levels which contribute strongly to the inactivation of the host immune system (via activation of Adenosine A2A receptors on host immune cells; see Leone R D, Lo Y C, and Powell J D, Comput Struct Biotechnol J. 2015 Apr. 8; 13:265-72). Our invention encompasses HD DHEA inhibition of the folate pathway, followed by folinic acid rescue. An optional way to replenish critical metabolites depleted during HD DHEA is to administer purines (e.g., adenine, guanine or hypoxanthine, their nucleosides or nucleotides) and a pyrimidine (e.g., uracil, its nucleoside or nucleotides), along with S-adenosylmethionine (SAMe). See FIG. 4.

Depletion of monoamines. A variety of monoamines are critical for normal physiology. These include serotonin (CAS 50-67-9), dopamine (CAS 51-61-6), melatonin (CAS 73-31-4), epinephrine (CAS 51-43-4) and norepinephrine (CAS 51-41-2). (See, for example, Perrier, J F and Cotel, F, Curr Opin Neurobiol. 2014 Dec. 29; 33C:1-7. doi: 10.1016/j.conb.2014.12.008). As illustrated in FIG. 7, depletion of BH4 suppresses synthesis of L-DOPA, from which dopamine, norepinephrine and epinephrine are subsequently synthesized. Replenishment of BH4 in DHEA-treated animals relieves this suppression, restoring normal or near normal dopamine, norepinephrine and epinephrine levels. However, a cost effective alternative to BH4 supplementation to relieve suppression of this pathway in DHEA-treated animals is to administer a reconstitution protocol that includes L-DOPA (CAS 59-92-7), Pyridoxine (Vitamin B6; pyridoxal 5′-phosphate; CAS 65-23-6), ascorbate (Vitamin C, CAS 50-81-7), and S-Adenosyl-methionine (SAMe; CAS 29908-03-0), albeit without alleviating phenylalanine build up, as BH4 supplementation does. However, this alternative protocol can lead to emesis in some dogs due to L-DOPA's systemic conversion to dopamine. Carbidopa (See Lotti, V J and Clark, C, Eur J Pharmacol. 1974 March; 25(3):322-5), domperidone (See Shuto, K et. al. (J Pharmacobiodyn. 1980 December; 3(12):709-14), and other dopamine antagonists can be added to prevent metabolism of L-DOPA to dopamine until it reaches the brain, thereby reducing or eliminating emesis caused by systemically administered (e.g., per os) L-DOPA.

Serotonin is found in the enterochromafin cells of the GI tract (approx. 90% of total body serotonin), platelets, and in the CNS. As illustrated in FIG. 6, serotonin and melatonin synthesis are dependent upon BH4, and their synthesis is therefore inhibited in DHEA-treated mammals. However, an alternative biosynthetic mechanism can be employed in which 5-hydroxytryptophan (5HT; CAS 4350-09-8), Vitamin C (Ascorbic acid; CAS 50-81-7), Pyridoxine (pyridoxal phosphate, Vitamin B6; CAS 54-47-7), vitamin B5 (pantothenic acid; CAS 599-54-2) and S-Adenosylmethionine (SAMe; CAS 29908-03-0 can be administered to produce both serotonin and melatonin. The present invention encompasses each of these methods to restore normal levels of dopamine, epinephrine, norepinephrine, melatonin and serotonin in DHEA-treated

The monoamine reconstitution protocol is thus a mixture of L-DOPA, 5HT, pyridoxine, SAMe, ascorbate, and pantothenic acid, and zinc, with or without the addition of a dopamine antagonist. See FIGS. 6 and 8.

For clarification, we will now enumerate our major findings underlying this invention.

Our studies show that administration of HD DHEA to cancer stricken dogs provides significant improvements in longevity, decreases in tumor volume, and increases in quality of life (when treatment is supplemented with specific metabolites, see below). In preliminary experiments we exposed human colonic carcinoma HT29SF cells to various doses of DHEA and assessed the effects of such treatment upon cell cycle kinetics. Results showed that DHEA arrested cells in the G1 phase of the cell cycle, mimicking the effects of the p53 tumor suppressor protein. Cell cycle arrest could be partly or completely reversed by addition of mevalonic acid and nucleosides (See FIG. 1).

Our studies show that BH4, IPA, NO, one carbon pool products (e.g., AMP, ADP, ATP), ubiquinone, and important monoamines are depleted, and phenylalanine levels are increased, in tissues of dogs administered HD DHEA.

Our studies show that HD DHEA to treat cancer can lead to an auto-inflammatory-like disorder which manifests itself in (1) a rash affecting skin, oral, ocular and other serosal surfaces; (2) intermittent fever; (3) monoamine depletion-related sequellae such as motor incoordination and depression; (4) lack of resistance to infections, especially in the ear and eye; (5) increased levels of phenylalanine in serum and tissues; (6) loss of pigmentation (melanin depletion); (7) neuropathy, particularly in the lower limbs, and (8) vasodilation problems attributable to the inability to synthesize NO.

Our studies show that reconstitution of BH4 and IPA (or mevalonate), addition of an NO donor such as potassium nitrate, addition of either ubiquinone and/or one or more tocotrienols, and addition of the monoamine reconstitution mix described above, with or without folinic acid rescue (or rescue by supplementation with the folate pathway products purines, pyrimidines, SAMe), abolish the negative sequellae of HD DHEA, permitting cancer to be treated in the absence of negative side effects.

### BRIEF DESCRIPTION OF FIGURES AND TABLES

FIGS. 1, 2. Flow cytometric analysis of HT-29SF, human colonic adenocarcinoma cells, exposed to HD DHEA. Cells were plated at 105/60-mm dish in duplicate. For analysis of cell cycle distribution, cultures were exposed to either 0, 25, 50, or 200 MMDHEA (FIG. 1). For analysis of reversal of cell cycle effects of DHEA (FIG. 2), cultures were exposed to either 0 or 25 MMDHEA, and the media were supplemented with MVA, CH, RN, MVA plus CH, or MVA plus CH plus RN or were not supplemented. Cultures were trypsinized following 0, 24, 48, or 72 h and fixed and stained using a modification of a procedure of Bauer et al. (14). Briefly, cells were collected by centrifugation and resuspended in cold phosphate-buffered saline. Cells were fixed in 70% ethanol, washed, and resuspended in phosphate-buffered saline. One ml hypotonic stain solution [50 Mg/ml propidium iodide (Sigma Chemical Co.), 20 ug/ml RNase A (Boehringer Mannheim, Indianapolis, Ind.), 30 mg/ml polyethylene glycol, 0.1% Triton X-100 in 5 mM citrate buffer] was then added, and after 10 min at room temperature, 1 ml of isotonic stain solution [propidium iodide, polyethylene glycol, Triton X-100 in 0.4 M NaCl] was added and allowed to stand for 30 min at 4° C. Total DNA content per cell was assessed by analysis of fluorescence at 585 A±21 nm using a FACScan flow cytometer, equipped with pulse width/pulse area doublet discrimination (Becton Dickinson Immunocytometry Systems, San Jose, Calif.). After calibration with fluorescent beads, a minimum of 2×IO4 cells/sample were analyzed, data were displayed as total number of cells in each of 1024 channels of increasing fluorescence intensity, and the resulting histogram was analyzed using the Cellfit analysis program (Becton Dickinson).

FIG. 3. Western blot analysis of the effect of DHEA on the membrane localization of the p21^{ras} oncoprotein. p21^{ras} is constitutively activated by mutation in a large percentage of human and canine cancers. In this study, human colonic HT29SF cells were treated for 24 hr with 50 uM DHEA or were not treated (Con). Cell lysates were separated by centrifugation into soluble (S) and particulate (P) fractions. The particulate fraction contains p21^{ras} oncoprotein that has been Isoprenylated and consequently has reached the inner surface of the plasma membrane, the only site from which it can exert its oncogenicity. The samples were resolved by SDS-PAGE and transferred onto a nitrocellulose membrane. p21^{ras} oncoprotein was detected with RAS 11 antibody as previously described (Schultz and Nyce, Cancer Research 51, 6563-6567, 1991). Arrow, position of p21^{ras} oncoprotein. Ordinate, migration of protein molecular weight markers (Mᵣ×10³). Results shown are representative of five independent experiments performed using the same method.

FIG. 4. Depletion and reconstitution of BH4, IPA and ubiquinone in dogs treated with HD DHEA. BH4 was assayed by the method of Niederwieser, A. et. al. (J Chromatogr. 1984 May 4; 290:237-46). For IPA quantitation, preliminary in vitro studies were performed in which DHEA-treated and control HT-29SF cells were exposed to [³H]-mevalonic acid (saponified; data not shown). Total RNA was then isolated using the guanidinium thiocyanate method, hydrolyzed to free bases and isolated on an Aminex A9 column, with UV detection, as previously described (Nyce, J et. al., Proc Natl Acad Sci USA. 1993 Apr. 1; 90(7):2960-4). Increased incorporation of [³H]-mevalonic acid into isopentenyladenosine peaks was interpreted as reduced pools of free unlabeled IPA into which the [³H]-IPA equilibrated with high specific activity. (See Shultz and Nyce, Cancer Research 51(24):6563-7, 1992). This was borne out in in vivo experiments in dogs treated with DHEA in which buffy coat lymphocytes were extracted and IPA quantitated with UV detection using the same HPLC procedure. In those in vivo studies, total pools of isopentenylated tRNA were indeed found to be depleted. Ubiquinone concentrations were quantitated from buffy coat samples by HPLC via the method of Abe, K. et. al., (J Nutr Sci Vitaminol (Tokyo). 1978; 24(6):555-67). For ease of presentation, pretreatment values for all 3 determinations (BH4, IPA and ubiquinone) were given a value of 1, with treatment values presented as their respective fraction thereof. Male and female dogs had blood or other specimens drawn pretreatment, and then following 90 days of 10 mg/kg DHEA±reconstitution protocol.

FIG. 5. Folate pathway, its heavy dependence upon NADPH, and the ability to bypass this NADPH dependence using folinic acid supplementation.

FIG. 6. Depletion of one carbon pool products during HD DHEA, and their reconstitution by folinic acid supplementation. Fifteen ml blood samples were drawn and the buffy coat was isolated by low speed centrifugation in the standard manner. Nucleotides were extracted from 10⁶ lymphocytes and quantitated via HPLC by the method of de Abreu, R A et. al. (J Chromatogr 227(1):45-52, 1982), with slight modifications. AMP, ADP and ATP were quantitated. In neutered dogs prior to treatment with DHEA, [AMP]/10⁶ lymphocytes was approx. 50 nM; ADP approx. 300 nM; ATP approx. 350 nM. For ease of presentation, pretreatment values are presented with a value of 1, with treatment values presented as their respective fraction thereof.

FIG. 7. Serotonin and melatonin biosynthetic pathway.

FIG. 8. Depletion of serotonin in DHEA-treated dogs (10 mg/kg daily for 90 days), and its reconstitution by supplementation with BH4 and/or the aforementioned monoamine precursor/cofactor mix. The procedure of D'Souza, L and Glueck, H (Thromb Haemost. 1977 Dec. 15; 38(4):990-1001) was used to quantitate serotonin in intestinal biopsies obtained from neutered male and female dogs. Ten mg of intestinal mucosa was analyzed for each animal. The average serotonin extracted and measured from 10 mg of intestinal mucosa was 22.5 umols for neutered male dogs pretreatment, and 20.25 umols for neutered female dogs pretreatment. Results are presented as percentage of values in pretreatment dogs, mean±SEM.

FIG. 9. Catecholamine biosynthetic pathway.

FIG. 10. Depletion of norepinephrine in urine of dogs undergoing HD DHEA, and their return to normal values with the addition of the monoamine reconstitution mix. Urinary free norepinephrine was quantitated by the method of Mell, L D and Gustafson, A B (Clin Chem 23(3): 473-6, 1977), using a Waters HPLC system with a C18 reverse phase column and UV detector. 24 hour urine was collected from neutered male and female animals in amber glass containment vessels. To 100 ml of urine was added 1 ml of concentrated HCL as preservative. Pretreatment values for urinary serotonin excretion in neutered males averaged ug/24 hr, and for neutered females, ug/24 hr. These same dogs were then administered DHEA (10 mg/kg)±the reconstitution regimen which included the monoamine mix described above. This treatment continued for 90 days, at which time further urine specimens were collected and assayed for norepinephrine. The data are reported as the mean±SEM.

FIG. 11. Increase in phenylalanine levels in DHEA-treated neutered dogs, and their reduction by supplementation with BH4 in the reconstitution protocol. Before treatment, 10-15 ml blood samples were taken from male and female neutered dogs, and plasma was collected by low speed centrifugation by the standard method. The method of Neckers, L M et. al. (Clin Chem 27(1):146-8, 1981) was used to quantitate plasma phenylalanine concentrations, with fluorometric detection (280 nm excitation, 330 nm emission), and a 10 ul injection volume.

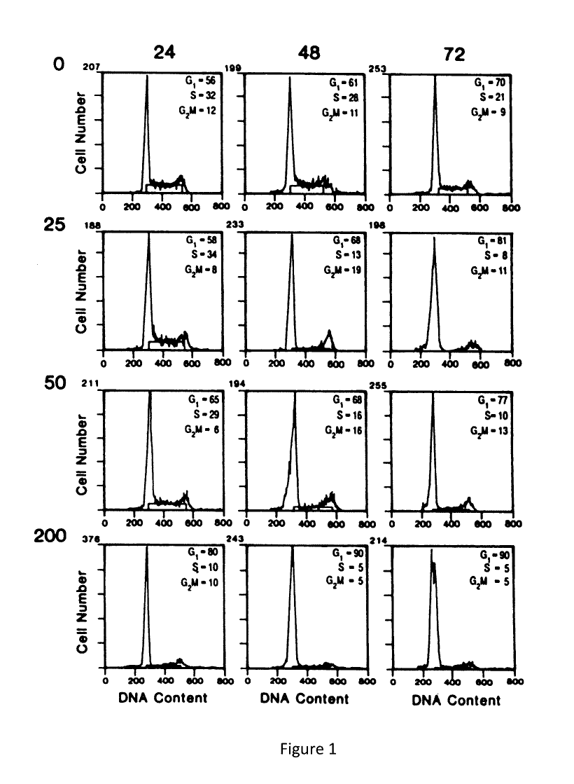

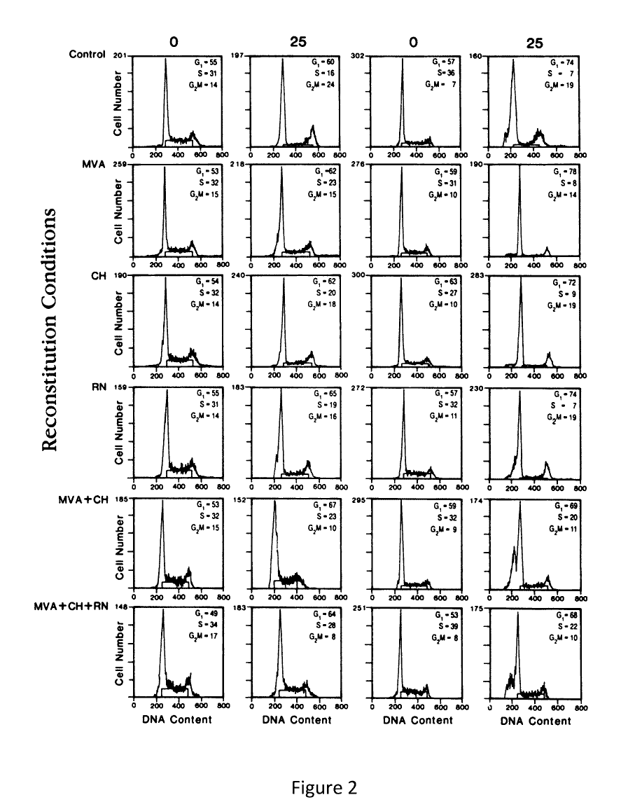

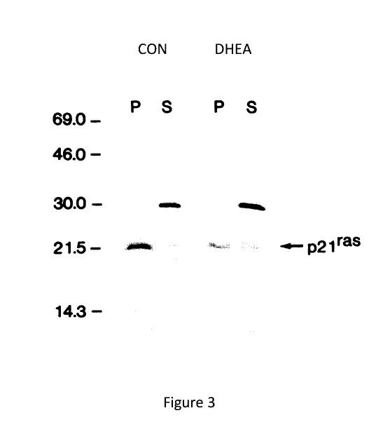

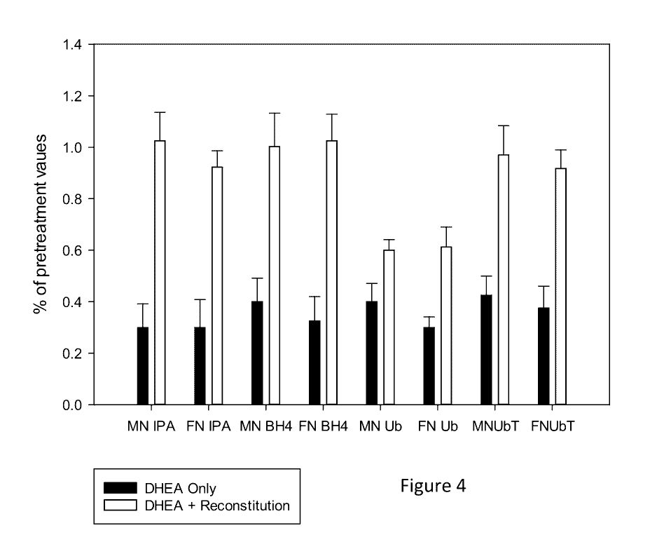

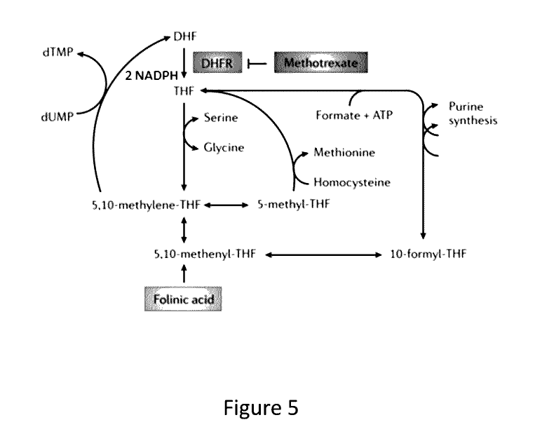

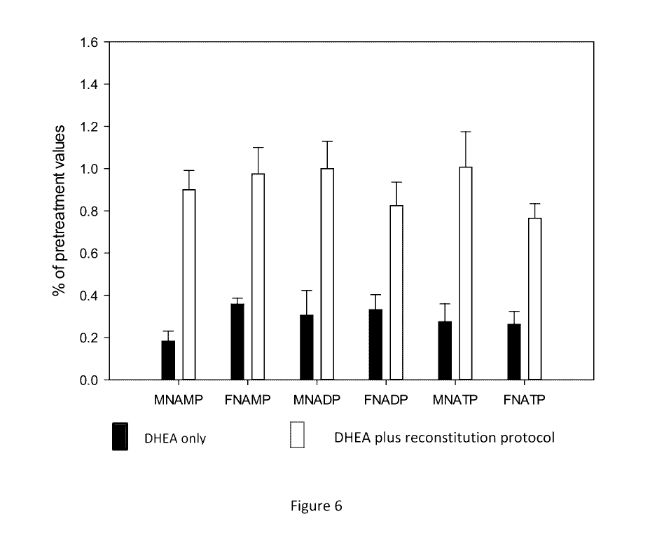

Table 1. Physical symptoms associated with HD DHEA-induced Nitric Oxide (NO) depletion. NO depletion was assessed by observation of effects (e.g., vasoconstriction leading to fluid retention in extremities, propensity to infection) known to be caused by NO depletion, and observing the elimination of these effects upon administration of a chemical nitric oxide donor (e.g., Potassium nitrate).

Table 2. Decreased tumor volume and increased longevity in dogs administered HD DHEA to treat nitrosamine-induced tumors. In this series of experiments, dogs were exposed to the organotropic carcinogen 1,2-dimethylhydrazine, ±HD DHEA as previously described (Nyce, J W et. al., Carcinogenesis February 1984; 5(1):57-62), except that DHEA did not commence until carcinogen exposure was complete. Organotropic carcinogens produce tumors in specific organs. Dimethylhydrazine produces exclusively colonic tumors, which metastasize to the liver. In this series of experiments 13 dogs were exposed to DMH only, and 13 were exposed to DMH followed by treatment with HD DHEA. Results show that although the number of tumors induced was not statistically different between control and HD DHEA-treated dogs, the weight of the individual colonic tumors, and especially the number and total weight of hepatic metastases was statistically significant (Student's t test, n, 0.05). Similar results were obtained using the organotropic carcinogens N-Nitrosodiethylamine (lung), dimethylnitrosamine (kidney), di-N-butylnitrosamine (urinary bladder), N-Nitroso-N-n-butylurea (lymphoma), N-methyl-N-Nitrosourea (mammary gland), and others.

Table 3. Decreased tumor volume, increased longevity (compared to historical controls), and improved quality of life in dogs with spontaneous cancers.

### DETAILED DESCRIPTION OF INVENTION

A preferred embodiment of the invention is a pharmaceutical composition of DHEA in sufficient amount to decrease tumor NADP(H) concentrations, and sufficient BH4, IPA, Potassium Nitrate (or similar nitric oxide donor), folinic acid (or purines, pyrimidines, SAMe), monoamine precursors and cofactors, and ubiquinone or tocotrienol or ubiquinone and tocotrienol to maintain or reconstitute normal or near normal levels of, respectively, BH4, IPA, nitric oxide, one carbon pool metabolism, monoamines, and ubiquinone, thereby preventing the negative side effects of HD DHEA. DHEA or any of its congeners may be used in combination. For example, DHEA might be combined with Fluasterone to modulate but not completely ablate the amount of steroid hormone synthesis that is possible, as DHEA is metabolized to steroid hormones, while Fluasteone is not. In terms of DHEA and its congeners which are also effective in inhibiting G6PD and thereby reducing NADPH levels, about 1 mg/kg to about 50 mg/kg, more preferentially 5 mg/kg to about 30 mg/kg, and most preferentially 10 mg/kg to 25 mg/kg represents a satisfactory dose for the purpose of this invention. DHEA, DHEAS, DHEA sulfatide and any of the salts and derivatives noted above may be manufactured and purified by any of several published methods. For example, the sulfatide can be prepared in high yield (68%) by the reaction of the silver salt of 5-androstene-3β-ol-17-one 3-sulfate with dipalmitoyl α-iodopropylene glycol (Abou-Gharbia, M et. al., J Pharmaceutical Sciences 70:10, 1154-1157, 1981). 7-Keto DHEA and its isomers can be prepared by any of several published procedures (See, for example, USPTO Application US 20070032462 A1). DHEA itself can be manufactured in high yield under mild reaction conditions using the procedure outlined in CN 102212099 A, and by many other methods. DHEA salicylate can be manufactured by the method described in U.S. Pat. No. 5,736,537 A. DHEA and its isomers, derivatives, precursors, metabolites, etc. useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; as injectables, and the like. DHEA or its isomers, derivatives, metabolites and precursors can be administered orally as a liquid, a lozenge, a capsule or a tablet. Flavor additives, stabilizers, solubilizing agents and flow enhancers and the like can be used to modify its flavor, activity, solubility and compressibility. DHEA or its isomers, derivatives, metabolites and precursors may also be administered non parenterally by any number of methods including transdermally; as a suppository; by inhalation of respirable formulations either to the lung or nasal cavities; by way of eye or drops; sublingually; and by injection by any of the following routes: subcutaneous, intravenous, intraperitoneal, intratumor, intracranial or intrathecal. Such injections can also be formulated as depot deliveries to increase duration, or to achieve another effect. DHEA or its isomers, derivatives, precursors or metabolites can be formulated as described above either alone or in combination with any or more of the other components of this invention, which include BH4, IPA, one or more nitric oxide donors, folinic acid, monoamine precursors and cofactors, ubiquinone and/or one or more tocotrienols.

BH4 can also be manufactured using any one of several methods. See, for example, U.S. Pat. No. 3,505,329 A; U.S. Pat. No. 8,178,670 B2: U.S. Pat. No. 4,595,752 A; CN101959891 A; WO2012048451 A1; CA2678165 C (Crystalline forms of the dihydrochloride); CN102443006 A (Hydrochloride); U.S. Pat. No. 4,649,197 A (sulfate); U.S. Pat. No. 4,550,109 A (lipoidal derivatives); WO2013152608 A1 (sapropterin dihydrochloride). Preparation of a sulfate of BH4 is described in CA 1250837 At BH4 and its isomers, derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. BH4 and its isomers, derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. BH4 and its isomers, derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. BH4 and its isomers, derivatives, and prodrugs can be formulated either alone or in combination with any or all of the other components of this invention, which include DHEA, IPA, one or more nitric oxide donors, folinic acid, monoamine precursors and cofactors, ubiquinone and/or one or more tocotrienols. For the purpose of this invention, BH4 can be administered at a dose of between 0.1 and 50 mg/kg, more preferentially between 1 and 30 mg/kg, and most preferentially between 5 and 25 mg/kg.

IPA can also be manufactured using any one of several methods including the classical Dimroth rearrangement, and by nucleophilic substitution reactions. (See, for example, Turner, M. L., Thesis, Department of Chemistry, Atlanta University, 1980; Robins, M. L. et. al., Biochemistry 6: 1837-1848, 1967; Rajabi, M. et. al., Nucleic Acid Therapeutics 21 (5), 2011). IPA and its isomers, derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. IPA and its isomers, derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. IPA and its isomers, derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. IPA and its isomers, derivatives, and prodrugs can be formulated either alone or in combination with any or all of the other components of this invention, which include DHEA, BH4, one or more nitric oxide donors, folinic acid (or purines, pyrimidines, SAMe), monoamine precursors and cofactors, ubiquinone and/or one or more tocotrienols. For the purpose of this invention, IPA can be administered at a dose of, preferentially, between 0.1 mg/kg and 50 mg/kg; more preferentially between 1 mg/kg and 30 mg/kg; and most preferentially between 2 mg/kg and 10 mg/kg.

Potassium nitrate is available commercially in human pharmaceutical grade. Other nitric oxide donors are also enveloped by our invention, including but not limited to furoxan-based DHEA hybrid molecules as described in the literature (See Huang, Y et. al., Steroids 2015, May 22, pii:S0039-128X(15)00149-X); sodium nitrite; nitric oxide gas; S-nitrosothiol, diazeniumdiolate; NONOate; furoxan; nitroaspirin; and organic nitrate (see Miller, M R and Megson, I L, Br J Pharmacol 151(3):305-321, June, 2007). Nitric oxide donors, derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Nitric oxide donors, derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Nitric oxide donors, derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Nitric oxide donors, derivatives, and prodrugs can be formulated either alone or in combination with any or all of the other components of this invention, which include DHEA, BH4, IPA, folinic acid, monoamine precursors and cofactors, ubiquinone and/or one or more tocotrienols. For the purpose of this invention, potassium nitrate can be administered at a dose of, preferentially, 1 mg/kg to 50 mg/kg; more preferentially 2 mg/kg to 25 mg/kg; and most preferentially at a dose of 5 mg/kg to 10 mg/kg.

Tocotrienols can be manufactured using any one of several methods including isolation from palm and other oils (Ng, M H et. al., Lipids. 2004 October; 39(10):1031-5; Luidy Rodriguez Posada et. al., Separation and Purification Technology Volume 57, Issue 2, 15 Oct. 2007, Pages 220-229), rice bran (Qureshi A A, et. al., J Agric Food Chem. 2000 August; 48(8):3130-40), and other sources (See N. Othman, et. al., 2010. Journal of Applied Sciences, 10: 1187-1191). The palmitate, stearate and 4-phenylbenzoate esters of D-gamma-tocotrienol can also be synthesized and purified using published procedures (See U.S. Pat. No. 5,670,668). Tocotrienols (α, β, γ, δ, separately or in any combination), their derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Tocotrienols (α, β, γ, δ, separately or in any combination), their derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Tocotrienols (α, β, γ, δ, separately or in any combination), their derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Tocotrienols (α,β,γ,δ, separately or in any combination), their derivatives, and prodrugs can be formulated either alone or in combination with any or all of the other components of this invention, which include DHEA, BH4, IPA, one or more nitric oxide donors, monoamine precursors and cofactors, and ubiquinone. For the purpose of this invention, tocotrientols, either individual isomers or in combinations, can be administered at a dose of preferentially, 1 mg/kg to 50 mg/kg; more preferentially at a dose of 5 mg/kg to 30 mg/kg; and most preferentially at a dose of 10 mg/kg to 25 mg/kg.

Ubiquinone is available in pharmaceutical grade purity or can be manufactured by any one of several methods. For example, optically pure ubiquinone can be synthesized in bulk by the process described in U.S. Pat. No. 6,506,915 B1, and U.S. Pat. No. 6,686,485 and a publication by Mahendra, M., et, al. (International Journal of Chemical Sciences and Research ISSN Print: 2249-0329 Website: http://www.ijcsr.co.in/). Both describe a semi synthetic procedure using solanesol derived from tobacco waste as the starting material for the sterospecific synthesis of ubiquinone. Additionally, large scale synthesis of ubiquinone in high yield can be performed by an SN2′-type nucleophilic displacement reaction between copper-catalyzed Grignard reagent and allylic acetate as reported by Wang, Fen et. al. (Letters in Organic Chemistry, Volume 3, Number 8 August 2006, pp. 610-612(3)). Ubiquinone, its derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Ubiquinone, its derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Ubiquinone, its derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Ubiquinone, its derivatives, and prodrugs can be formulated either alone or in combination with any or all of the other components of this invention, which include DHEA, BH4, IPA, one or more nitric oxide donors, monoamine precursors and cofactors, and one or more Tocotrienols. For the purpose of this invention, ubiquinone can be administered at a dose of, preferentially, 0.1 mg/kg to 20 mg/kg; more preferentially 0.5 mg/kg to 10 mg/kg; and most preferentially, 1 mg/kg to 5 mg/kg.

Folinic acid can be synthesized and purified by many methods in the public domain, including that of Temple, C, Jr et. al. (J Med Chem. 22(6):731-4, 1979; Sato, J K et. al., Anal Biochem. 154(2):516-24, 1986; U.S. Pat. No. 5,134,235 A), and by methods still under patent, for example U.S. Pat. No. 8,633,202 B2. As synthesized folinic acid exists as a mixture of optical isomers, these optical isomers can be separated by the method described in U.S. Pat. No. 5,599,931 A, and by other methodologies. Stabilized aqueous preparations of folinic acid, for example, for injection, can be obtained through the method described in U.S. Pat. No. 6,613,767 B1 and European Patent EP1640008 B1. The sodium salt of folinic acid can be prepared according to the method described in U.S. Pat. No. 6,160,116 A. Folinic acid, its derivatives, salts and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Folinic acid, its derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Folinic acid, its derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Folinic acid, its derivatives, and prodrugs can be formulated either alone or in combination with any or all of the other components of this invention, which include DHEA, BH4, IPA, one or more nitric oxide donors, monoamine precursors and cofactors, ubiquinone and/or one or more Tocotrienols. For the purpose of this invention, folinic acid can be given in a dose range of, preferentially, 0.01 mg/kg to about 15 mg/kg; more preferentially 0.1 mg/kg to 10 mg/kg; and most preferentially 0.3 mg/kg to 5 mg/kg.

Monoamine precursors and cofactors, including L-DOPA, 5HT, pyridoxine, SAMe, ascorbate, and pantothenic acid, and zinc, can each be purchased commercially in highly purified form, or can be synthesized and purified by many methods in the public domain. Highly purified L-DOPA is available commercially, and can be manufactured according to standard chemical methods employing asymmetric synthesis and metal catalysts, or electroenzymatically employing a tyrosinase-immmobilized cathode under the reduction potential of DOPAquinone (See Min, K et al, J. Biotechnol 146(1-2):40-44, 2010). 5-HT is available commercially in highly purified form. It can be synthesized by many published methods (See, for example, Frangatos, G and Chubb, F, Can J Chem 37:1374-76, 1959), including a newly reported cofactor regeneration process using modified L-phenylalanine 4-hydroxylase of Chromobacterium violaceum. (See Hara, R and Kino, K, AMB Express 3:70, 2013, doi:10.1186/2191-0855-3-70). Pyridoxal phosphate is available commercially in highly purified form. SAMe is available commercially in highly purified form, of which the dihydrochloride salt is particularly useful. Ascorbic acid is also available commercially in highly purified form, as are pantothenic acid and various formulations of zinc. Each of these monoamine precursors or cofactors, their derivatives, salts and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Each of these monoamine precursors or cofactors, their derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Each of these monoamine precursors or cofactors, their derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Each of these monoamine precursors or cofactors, their derivatives, and prodrugs can be formulated either alone or in combination with any or all of the other components of this invention, which include DHEA, BH4, IPA, one or more nitric oxide donors, folinic acid (or adenine (or hypoxanthine) and uracil, their nucleosides or nucleotides), ubiquinone and/or one or more Tocotrienols. For the purpose of this invention, L-DOPA can be administered at a dose of, preferentially, from 0.1 mg/kg to 25 mg/kg; more preferentially, 0.5 mg/kg to 15 mg/kg; and most preferentially from 1 mg/kg to 10 mg/kg. For the purpose of this invention, 5-HT can be administered at a dose of, preferentially, 0.1 mg/kg to 25 mg/kg; more preferentially, 0.5 mg/kg to 15 mg/kg; and most preferentially, 1 mg/kg to 10 mg/kg. For the purpose of this invention, pyridoxine can be administered at a dose of, preferentially, 0.05 mg/kg to 5 mg/kg; more preferentially, 0.09 mg/kg to 2.5 mg/kg; and most preferentially, 0.5 mg/kg to 1 mg/kg; For the purpose of this invention, SAMe can be administered at a dose of, preferentially, 1 mg/kg to 100 mg/kg; more preferentially, 2 mg/kg to 50 mg/kg; and most preferentially, 5 mg/kg to 10 mg/kg. For the purpose of this invention, ascorbic acid can be administered at a dose of, preferentially, 1 mg/kg to 100 mg/kg; more preferentially, 2 mg/kg to 50 mg/kg; and most preferentially, 5 mg/kg to 25 mg/kg. For the purpose of this invention, pantothenic acid can be administered at a dose of, preferentially, 0.1 mg/kg to 100 mg/kg; more preferentially, 0.5 mg/kg to 50 mg/kg; and most preferentially, 1 mg/kg to 10 mg/kg. For the purpose of this invention, Zinc may be administered at a dose of, preferentially, 0.05 mg/kg to 10 mg/kg; more preferentially, 0.1 mg/kg to 5 mg/kg; and most preferentially, 0.5 mg/kg to 2.5 mg/kg.

As used in the context of this invention, co-administration refers to temporal proximity. Thus, the agents described as “co-administered” may be administered exactly together; they may be delivered one or more before the other(s), so as to prevent the onset of negative side effects; they may be delivered one or more after the other(s), for example, to cost effectively supply them only when the need becomes more pressing; or some combination of the above.

Examples of binders are gum tragacanth, acacia, starch, gelatine, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding poly-ester-polyamide-co-polymers, polyanhydrides, polyorthoestens, polyphosphazene and poly-carbonates. The biological degradable polymers may be linear, branched or crosslinked.

Specific examples are poly-glycolic acid, poly-lactic acid, and poly-d,l-lactide/glycolide. Other examples for polymers are water-soluble polymers such as polyoxalkylenes (polyoxethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol and esters or -ethers thereof, poly-vinylimidazole, polyvinylpyrrolidon, and natural polymers like chitosan.

Examples for excipients are phosphates such as dicalcium phosphate.

Examples for lubricants are natural or synthetic oils, fats, waxes, or fatty acid salts like magnesium stearate, sesame oil, olive oil, coconut oil, tocopherols and the like.

Surfactants may be ionic, anionic, amphoteric or neutral. Examples for surfactants are lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylamino-ethane-2-sulfonic acid, and taurocholio acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Examples for sweetening agents are sucrose, fructose, lactose, sodium saccharine, Steviol glycosides, or aspartame.

Examples for flavoring agents are bacon, beef, chicken, peanut butter, oil of wintergreen or fruit flavors like cherry or orange flavor.

Examples for coating materials are gelatin, wax, shellac, sugar or biological degradable polymers.

Examples for preservatives are methyl or propylparabens, sorbic acid, chlorobutanol, sodium nitrite, potassium nitrate, phenol, butylated hydroxytoluene, butylated hydroxyanisole and thimerosal.

Examples for thickeners are synthetic polymers, fatty acids and fatty acid salts and esters and fatty alcohols.

Examples for antioxidants are vitamins, such as vitamin A, vitamin C, vitamin D or vitamin E, tocopherols, tocotrienols, quercetin, curcumin, L-cysteine, L-acetyl cysteine, sesamin, sesamol, vegetable extracts or fish oils.

Examples for liquid carriers are water, alcohols such as ethanol, glycerol, propylene glycol, liquid polyethylene glycols, triacetin and oils. Examples for solid carriers are talc, clay, micro-crystalline cellulose, silica, alumina and the like.

The formulation according to the invention may also contain isotonic agents, such as sugars, buffers or sodium chloride.

A syrup or elixir may contain the polymorph of the invention, sucrose or fructose as sweetening agent, a preservative like methylparaben, a dye and a flavoring agent.

Slow release formulations may also be prepared from the polymorph according to the invention in order to achieve a controlled release of the active agent in contact with the body fluids in the gastro intestinal tract, and to provide a substantial constant and effective level of the active agent In the blood plasma. The formulation may be embedded for this purpose in a polymer matrix of a biological degradable polymer, a water-soluble polymer or a mixture of both, and optionally suitable surfactants. Embedding can mean in this context the incorporation of microparticles in a matrix of polymers. Controlled release formulations are also obtained through encapsulation of dispersed micro-particles or emulsified micro-droplets via known dispersion or emulsion coating technologies.

### UNITED STATES PATENTS CITED

U.S. Pat. No. 5,567,696 A, 1996, McGuire, J L, Van Vollenhoven, R F, and Engelman, E G, Treatment of systemic lupus erythematosus with dehydroepiandrosterone
U.S. Pat. No. 7,893,044 B2, 2011, Nyce, Jonathan W. Composition and method for altering levels of or sensitivity to adenosine with analogs of dehydroepiandrosterone
U.S. Pat. No. 5,527,789 A, 1996, Nyce, Jonathan W. Method of inhibiting carcinogenesis by treatment with dehydroepiandrosterone and analogs thereof
U.S. Pat. No. 6,087,351, 2000, A Nyce, Jonathan W, Method for reducing adenosine levels with a dehydroepiandrosterone and optionally a ubiquinone
U.S. Pat. No. 6,670,349 B1, 2003, Nyce, Jonathan, Composition & method for altering levels of or sensitivity to adenosine with a dehydroepiandrosterone &/or a ubiquinone
U.S. Pat. No. 7,456,161 B2, 2008, Jonathan W Nyce, Use of DHEA and DHEA-sulfate for the treatment of chronic obstructive pulmonary disease
U.S. Pat. No. 5,134,235 A, 1992, Mueller, H R et al, Process for separating folinic acid
U.S. Pat. No. 8,633,202 B2, Jequier, P, 2014, Crystalline levofolinic acid and process for its preparation
U.S. Pat. No. 5,599,931 A, Ripa, G et. al., 1997, Process for separating stereoisomers of folinic acid
U.S. Pat. No. 6,613,767 B1, Nijkerk, A J and Vermeer, J M P, 2003, Stable aqueous folinate solution
U.S. Pat. No. 6,160,116 A, Mueller, H R et al., 2000, Sodium salt of (6S)-folinic acid

### FOREIGN PATENTS CITED

WO 1996025164 A1, 1995 Use of dehydroepiandrosterone to treat depression. Goodyer, Ian Michael, and Joseph, Herbert (ABSTRACT: Dehydroepiandrosterone (DHEA) is indicated in the treatment of depression. Clinical data show a statistically significant association between blood levels of DHEA, abnormally low (which can be determined by measuring the salivary DHEA), especially in the morning, and major depression, diagnosed based on recognized clinical criteria. The administration of DHEA increased serum levels of DHEA).
WO 1998032445 A1, 1998, Brian Berger, Samuel S C Yen, Use of dehydroepiandrosterone to treat primary adrenal insufficiency and Addison's disease (ABSTRACT: The present invention provides a method of treating an individual with primary adrenal insufficiency, comprising the step of administering an effective amount of dehydroepiandrosterone to said individual. Also provided is a method of treating an individual with adrenal failure secondary to dysfunctions of the hypothalamus and/or pituitary gland, comprising the step of administering an effective amount of dehydroepiandrosterone to said individual and a method of treating an individual with adrenal insufficiency due to acquired human immunodeficiency syndrome (AIDS), comprising the step of administering an effective amount of dehydroepiandrosterone to said individual).
WO 2002043737 A1, Zenk, J L and Zenk, R, 2002, Treatment of chronic fatigue syndrome and fibromyalgia syndrome (ABSTRACT: Chronic fatigue syndrome (CFS) and fibromyalgia syndrome (FMS) can be treated by the administration of Δ5-androstene-3β-ol-7,17 dione and metabolizable precursors thereof).
WO 2003002123 A3, Van Der Hoop, Roland Gerritsen, 2003, Therapeutic combinations for the treatment of hormone deficiencies (ABSTRACT: The present invention relates to methods of treating, preventing, or reducing the risk of developing a male or female menopause disorder or symptom in a mammal by administering to the mammal a sex hormone binding globulin synthesis inhibiting agent and one or more steroids, including, for example, an androgen or an estrogen; to combinations for treating, preventing or reducing the risk of developing a male or female menopause disorder or symptom in a mammal; and to compositions for treating, preventing, or reducing the risk of developing a male or female menopause disorder or symptom in a mammal, where the composition comprises a sex binding globulin synthesis inhibiting agent and one or more steroids, including, for example, an androgen or an estrogen. In addition, the methods, combinations and compositions may be used in conjunction with other pharmaceutical agents aimed at improving sexual performance or impotence, increasing libido, or treating erectile dysfunction, such as VIAGRA®, to enhance their effectiveness.
EP 1640008 B1, Niyaji, T N P et. al., 2011, A stabilized aqueous (6S)-folinic acid preparation for injection).

### OTHER CITATIONS

Aas, T, et. al., Nat Med. 1996 July; 2(7):811-4
Abe, K. et. al., (J Nutr Sci Vitaminol (Tokyo). 1978; 24(6):555-67
Ahlem C N, et. al., Steroids. 2011 June; 76(7):669-74
Aizawa K et al, Am J Physiol Endocrinol Metab. 2007 February; 292(2):E577-84. Epub 2006 Oct. 3
Basualto-Alarcón C, et al, Front Endocrinol (Lausanne). 2014 Dec. 18; 5:217
Baliga, R S, et al, Respiratory and Critical Care Medicine, Abstract Issue, B63, Experimental models in pulmonary hypertension, American Thoracic Society International Conference Abstracts, 2012
Bélanger B. et. al., Journal of Steroid Biochemistry 32 695-698, 1989
Bentinger, M et. al. (Biofactors. 2008; 32 (1-4):99-111
Bifulco, M. Malfitano, A M, Proto, M C et al. Anticancer Agents Med Chem 2008, 8(2): 2000-2004
Borsi, J D et al, Pediatr Hematol Oncol. 1990; 7(4):347-63; folinic acid CAS 1492-18-8
Callewaert F, Boonen S, Vanderschueren D. Trends Endocrinol Metab. 2010 February; 21(2):89-95
Caso, Francesco et al., Int J. Rheumatol 2013, Oct. 24
Chamouni A, Oury F, Arch Biochem Biophys. 2014 Nov. 1; 561:147-53
Chan, K T and Lung, M L, Cancer Chemother Pharmacol. 2004 June; 53(6):519-26. Epub 2004 Mar. 4
Coleman, J W, Int Immunopharmacol. 2001 August; 1(8):1397-406
Cunningham G R, Asian J Androl. 2015 March-April; 17(2):192-6
Cutler G B Jr, et al., Endocrinology. 1978 December; 103(6):2112-8
de Abreu, R A et. al., J Chromatogr 227(1):45-52, 1982
Dillon, J S, Curr Drug Targets Inflamm Allergy. 2005 June; 4(3):377-85
D'Souza, L and Glueck, H (Thromb Haemost. 1977 Dec. 15; 38(4):990-1001
Hayashi F., Carcinogenesis. 1994 October; 15(10):2215-9
Hofmann and Mostl (J Reprod Fertil Suppl 57:67-70, 2001
Hudson, T. S. et al, Carcinogenesis 33(6):1225-1230, 2012
Huneeus, V Q, Wiley, M H and Siperstein, M D, PNAS USA October 1980: 77(10):5842-5846
Huneeus, V Q, Wiley, M H and Siperstein, M D, PNAS USA October 1980: 77(10):5842-5846
Inoue, Takayoshi et al, Mol Cell Endocrinol 2012 Oct. 22; 362(1-2):19-28. Epub 2012 May 22
Kapatos, G., The neurobiology of tetrahydrobiopterin biosynthesis, IUBMB Life 65(4):323-33, 2103
Khorram, O et al, J Gerontol A Biol Sci Med Sci. 1997 January; 52(1):M1-7
Khorrami A, et. al., Drug Res (Stuttg). 2014 April; 64(4):177-81. doi: 10.1055/s-0033-1354374. Epub 2013 Sep. 11
Koike, H. et al Neurology. 84(10):1026-33, 2015; doi: 10.1212/WNL.0000000000001343. Epub 2015 Feb. 6
Kryukov, G. V. et al, J. Biol. Chem. 274, 33888-33897, 1999
Labrie F, Dupont A & Bélanger A. In Important Advances in Oncology, pp 193-217, 1985. Eds V T de Vita, S Hellman & S A; Rosenberg. Philadelphia: J. B. Lippincott
Labrie F, Belanger A, Cusan L & Candas B, Journal of Clinical Endocrinology and Metabolism 82:2403-2409
Labrie, F et al, Front Neuroendocrinol. 2001 July; 22(3):185-212
Laten, H M and Zahareas-Doktor, S PNAS USA February 1985; 82(4):1113-1115
Leone R D, Lo Y C, and Powell J D, Comput Struct Biotechnol J. 2015 Apr. 8; 13:265-72. doi: 10.1016/j.csbj.2015.03.008. eCollection 2015
Liu Q et al, Am J Pathol. 2012 August; 181(2):452-62
Lotti, V J and Clark, C, Eur J Pharmacol. 1974 March; 25(3):322-5
Mattmiller, S A et al, J. Nut Biochem 25(6):647-54, 2014
Mell, L D and Gustafson, A B (Clin Chem 23(3): 473-6, 1977
Mongillo P et al, Res Vet Sci. 2014 February; 96(1):33-8
Morgentaler A, Asian J Androl. 2015 January-February; 17(1):26-31
Mukherjee, Siddhartha, The Emperor of All Maladies, a Biography of Cancer; Simon & Schuster, 2010
Nagasakin, Y. et al., Pediatr Res. 1999 April; 45(4 Pt 1):465-73
Nakamaru-Ogiso E et. al., J Bioenerg Biomembr. 2014 August; 46(4):269-77. doi: 10.1007/s10863-014-9557-9. Epub 2014 Jul. 31
Neckers, L M et. al. (Clin Chem 27(1):146-8, 1981
Ney D M, Blank R D, and Hansen K E, Curr Opin Clin Nutr Metab Care. 2014 January; 17(1):61-8
Niederwieser, A. et. al. (J Chromatogr. 1984 May 4; 290:237-46
Nyce, J et. al., Proc Natl Acad Sci USA. 1993 Apr. 1; 90(7):2960-4
Nyce J W et al, Carcinogenesis. 1984 January; 5(1):57-62
Odell W D, Parker L N, Endocr Res. 1984-1985; 10(3-4):617-30
Olekhnovitch, R et al, J. Clin Invest 124(4):1711-1722, 2014
Oury F, Ann N Y Acad Sci. 2012 July; 1260:1-7
Pan, L et al, Brit Med J Case Rep bcr0320113927, 2011
Perrier, J F and Cotel, F, Curr Opin Neurobiol. 2014 Dec. 29; 33C:1-7. doi: 10.1016/j.conb.2014.12.008
Polimeni, M et. al., Biochem. J. 2011, 439, 141-149
Prudhomme, M, Recent Pat Anticancer Drug Discov 1(1):55-68, 2006
Rohrmann, Sabine et al, Clin Endocrinol (Oxf) 2011 August, 75(2):232-239
Rissetto, K et al, J Am Anim Hosp Assoc. 2011 January-February; 47(1):28-36
Rohrmann, S., et. al., Clin Endocrinol (Oxf). 2011 August; 75(2): 232-239. doi: 10.1111/j.1365-2265.2011.04043.x
Salerni, S. et al, Eur J Clin Invest. 2015 Apr. 3
Sathish V, Martin Y N, Prakash Y S, Pharmacol Ther. 2015 Jan. 14
Seeher, S. et al, Biochem J. 462(1):67-75, 2014
Stot, Crister A, Karel Dolezal, Anders Nordstrom et al. PNAS USA Dec. 19, 2000; 97(26):14778-14783
Scriver, C R and Clow, C L, Annu Rev Genet. 1980; 14:179-202
Sanford, Mark; Keating, Gillian M. (2009). “Sapropterin”. Drugs 69 (4): 461-76 doi:10.2165/00003495-200969040-00006. PMID 19323589
Sharma, R and Davidoff, M N, Oxidative stress and endothelial dysfunction in heart failure, Congest Heart Fail. 2002 May-June; 8(3):165-72
Schalinske1, K L and Steele, R D Carcinogenesis vol. 17 no. 8 pp. 1695-1700, 1996
Shuto, K et. al. (J Pharmacobiodyn. 1980 December; 3(12):709-14
Schoneshofer and Weber (J Clin Chem Clin Biochem 21(4):231-6, 1983
Shultz and Nyce, Cancer Research 51(24):6563-7, 1992
Sorenmo K U, Goldschmidt M, Shofer F, Ferrocone J. Vet Comparative Oncology. 2003 March; 1 (1): 48
Sorwell, K G and Urbanski, H F, J. Neuroendocrin 2013, 25(11):1062-1069, November 2013
Spinola M et al, Oncogene. 2005 Aug. 18; 24(35):5502-9
Srinath, R et al, J Clin Endocrinol Metab. 2015 April; 100(4):1602-8
Strisciuglio P and Concolino D, Metabolites. 2014 Nov. 4; 4(4):1007-17. doi: 10.3390/metabo4041007
Teske E, Naan E C, van Dijk E M, vanGarderen E, Schalken J A. Mol Cell Endocrinol. 2002 Nov. 29; 197(1-2):251-255
Torres de la Riva, G et al, PLoS One, 2013, 8(2):e55937
Tsuji, P. A. et al, PLoS One, 2015, 10(4):e0124487
Tsukamoto N, et. al., Blood Cells Mol Dis. 1998, 24(2):231-8
van der Burgh, R et al, Clin Immunol 147(3):197-206, 2013
van der Molen, H J, et al. Biochim Biophys Acta, 1971 Nov. 5; 248(2):343-62
van Spronsen F J¹, Enns G M, Mol Genet Metab. 2010; 99 Suppl 1:S90-5. doi: 10.1016/j.ymgme.2009.10.008
Villamil, J A, et. al., J Cancer Epidemiol 2009:591753
Widstrom, Richard L. Seminars in Reproductive Medicine December 2004; 22(4):289-98; Labrie, F et. al. Journal of Endocrinology (2005) 187, 169-196
Williams, John, Lipids, April 2000; 35(3):325-31

The embodiments of the invention in which an exclusive property or privilege is claimed are defined as follows:

1. A method for treating cancer in mammals comprising administration of High Dose (HD) Dehydroepiandrosterone or its congeners in amounts sufficient to deplete tumor NADP(H), and a reconstitution mixture to prevent the toxic side effects of NADP(H) depletion in normal cells, tissues and organs.

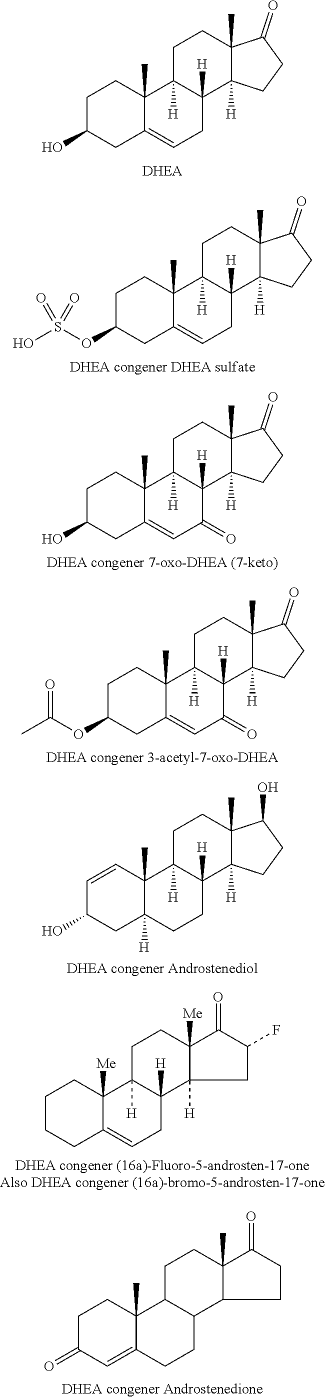

2. The substance(s) of claim 1 administered as an oral, injectable (intravenous, intratumor, subcutaneous, intraperitoneal, intracranial or intrathecal), inhalable, aerosolized, respirable, sublingual, topical (including ophthalmic, otic), transdermal, or suppository, in amounts sufficient to treat cancer in a mammal in need of such treatment.

3. A method for preventing side effects of HD DHEA performed according to the method of claim 1, comprising Tetrahydrobiopterin (BH4, Formula 3) or sepiapterin (Formula 3b), or pharmaceutically acceptable salts thereof, as a component of the reconstitution mixture co-administered with the substance(s) of claim 1, in amounts sufficient to inhibit the side effects of BH4 depletion in normal tissues.

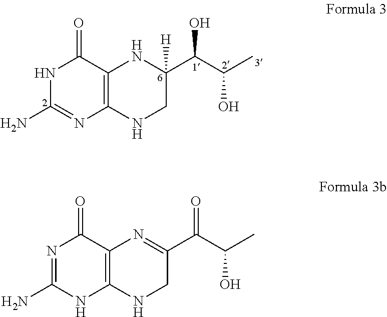 [SMILES: C[C@H](O)[C@H](O)[C@H]1CNc2nc(N)[nH]c(=O)c2N1 ; C[C@H](O)C(=O)C1=Nc2c([nH]c(N)nc2=O)NC1]

4. The substance(s) of claim 3 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent side effects of BH4 depletion in normal tissues during HD DHEA performed according to the method of claim 1, in a mammal in need of such treatment.

5. A method for preventing side effects of HD DHEA performed according to the method of claim 1, comprising N6-Isopentenyladenosine (IPA, Formula 5), its 5′-monophosphate or thioether (Formula 5b) or pharmaceutically acceptable salts thereof, as a component of the reconstitution mixture co-administered with the substance(s) of claim 1, in amounts sufficient to restore normal levels of isopentenylated tRNA in normal tissues.

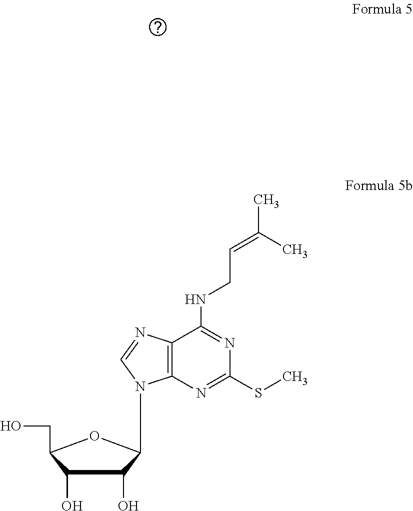

6. The substance of claim 5 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent side effects of IPA depletion in normal tissues during HD DHEA performed according to the method of claim 1, in a mammal in need of such treatment.

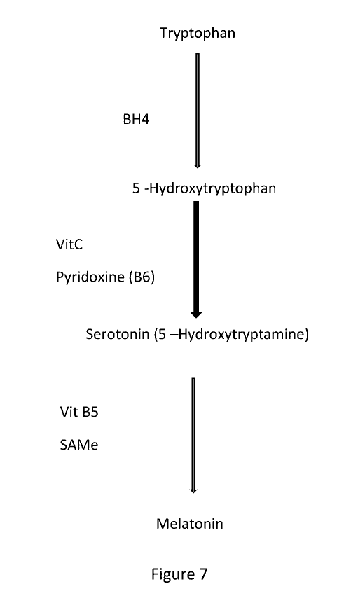

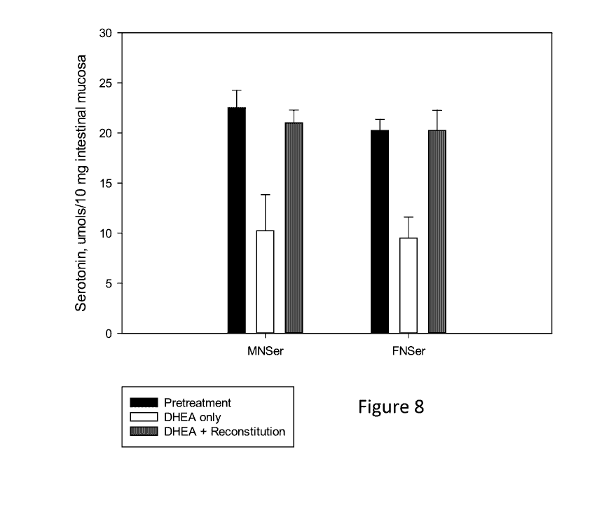

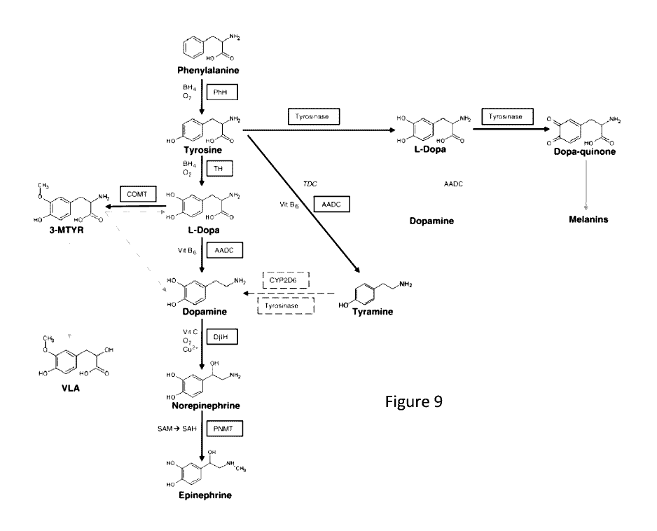

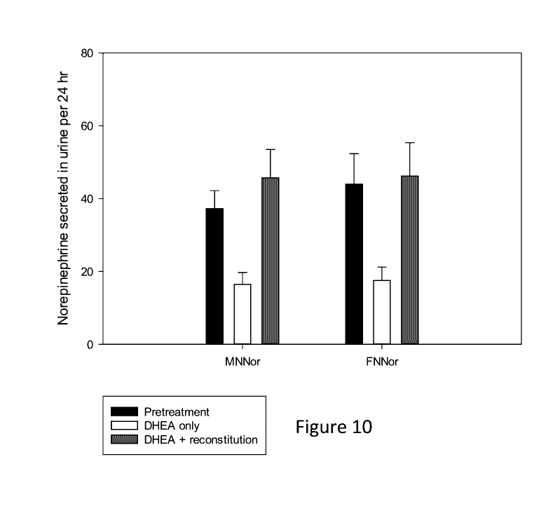

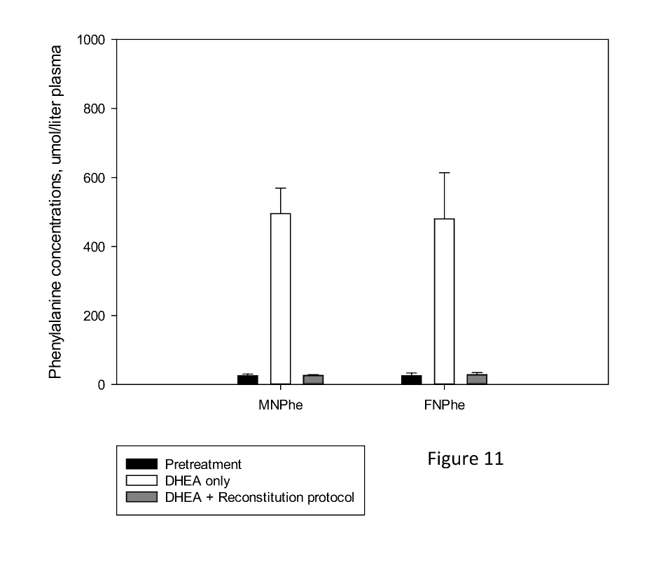

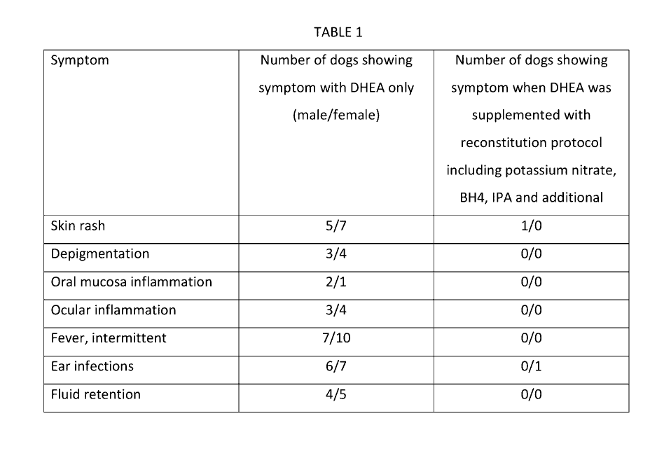

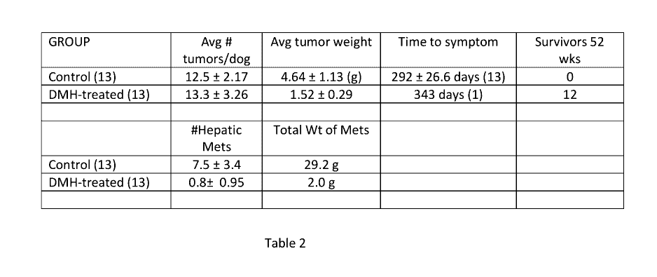

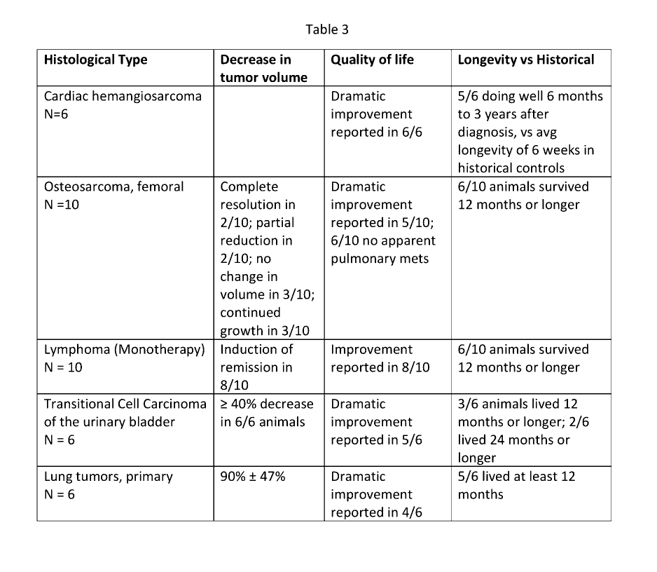

7. A method for preventing side effects of HD DHEA performed according to the method of claim 1, comprising a nitric oxide donor, e.g., potassium nitrate (KNO3, FIG. 7) or other nitric oxide donor, e.g., sodium nitrite (NaNO2, Formula 7b) as a component of the reconstitution mixture co-administered with the substance(s) of claim 1, in amounts sufficient to restore healthy levels of nitric oxide to normal tissues.

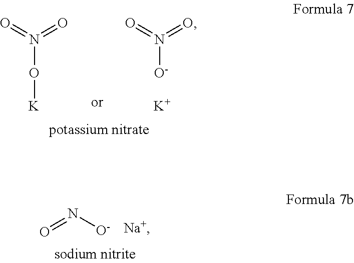

8. The substance of claim 7 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent side effects of nitric oxide depletion in normal tissues during HD DHEA performed according to the method of claim 1, in a mammal in need of such treatment.

9. A method for preventing depletion of ubiquinone during HD DHEA performed according to the method of claim 1, comprising alpha, beta, gamma and/or delta Tocotrienol (Formula 9) as a component of the reconstitution mixture co-administered with the substances of claim 1, in amounts sufficient to maintain healthy amounts of ubiquinone in normal tissues.

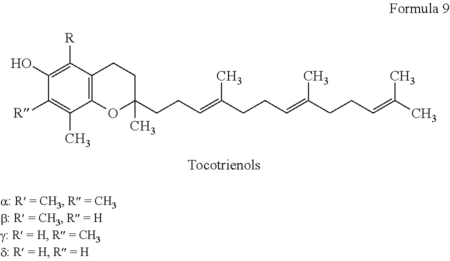

10. The substance(s) of claim 9 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent side effects of ubiquinone depletion in normal tissues during HD DHEA performed according to the method of claim 1, in a mammal in need of such treatment.

11. A method for preventing depletion of ubiquinone during HD DHEA according to the method of claim 1, comprising administration of ubiquinone (formula 11, chain length 6-10), or ubiquinol (formula 11b, chain length 6-10) as a component of the reconstitution mixture co-administered with the substances of claim 1, in sufficient amounts to inhibit side effects of ubiquinone depletion in normal tissues.

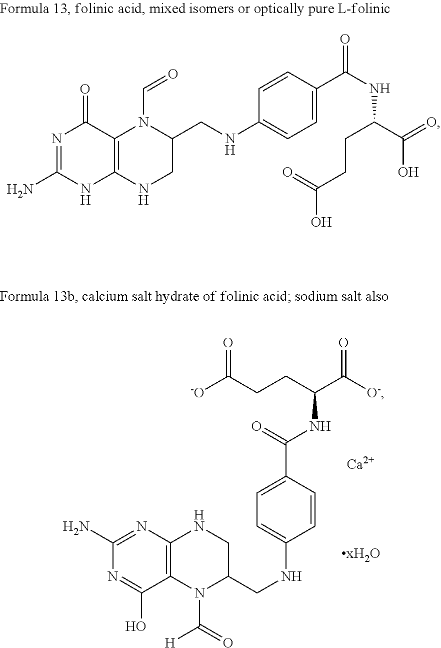

12. The substance of claim 11 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent side effects of ubiquinone depletion in normal tissues during HD DHEA performed according to the method of claim 1, in a mammal in need of such treatment.

13. A method for preventing depletion of products of the folate pathway during HD DHEA performed according to the method of claim 1, comprising administration of folinic acid (formula 8, mixed isomers or purified L-isomer) as a component of the reconstitution mixture co administered with the substance(s) of claim 1 in sufficient amounts to inhibit side effects of depletion of folate pathway products in normal tissues.

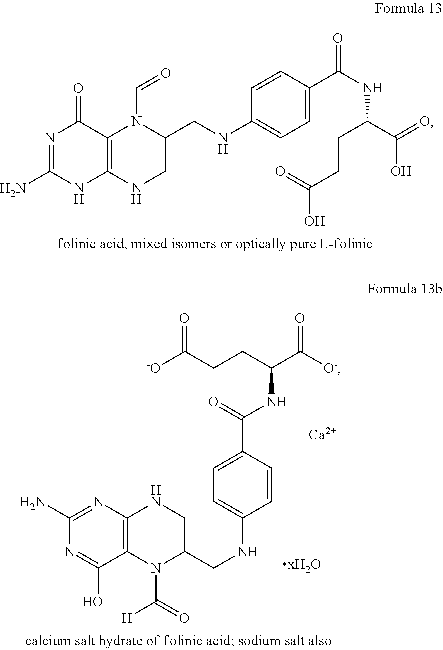

14. The substance(s) of claim 13 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent side effects of folate pathway product depletion in normal tissues during HD DHEA performed according to the method of claim 1, in a mammal in need of such treatment.

15. A method for preventing depletion of monoamines during HD DHEA performed according to the method of claim 1, comprising administration of the monoamine precursors 5-Hydroxtryptophan (5-HT; formula 15), pyridoxine (formula 15b), L-DOPA (formula 15c), S-adenosylmethionine (SAMe; Formula 15d), ascorbate (ascorbic acid; formula 15e), pantothenic acid (formula 15f), zinc, as components of the reconstitution mixture co administered with the substance(s) of claim 1 in sufficient amounts to maintain healthy levels of monoamines in normal tissues.

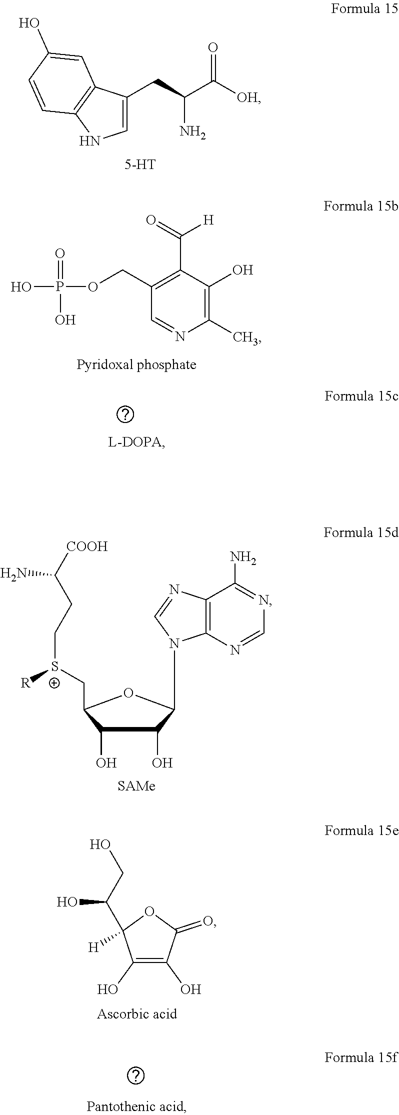

16. The substances of claim 15 administered as oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent monoamine depletion in normal tissues during HD DHEA performed according to the method of claim 1, in a mammal in need of such treatment.

17. A method for method for treating cancer without inducing side effects of such treatment, comprising administration of the substance(s) of claim 1, the substance(s) of claim 3, the substance(s) of claim 5, the substance(s) of claim 7, the substances in claim 9, the substances of claim 11, the substances of claim 13, and the substances of claim 15, in any combination and at any time that reduces or eliminates the negative side effects of HD DHEA performed according to the method of claim 1.

18. The method of claim 1 in which the substance(s) of claim 1 are administered intermittently, according to a schedule that avoids some or all of the negative side effects of HD DHEA performed according to the method of claim 1.

19. The method of claim 13 in which adenosine, adenosine monophosphate, adenosine diphosphate or adenosine triphosphate are administered in addition to folinic acid to remediate adenosine depletion at local sites.

20. The method of claim 5 in which IPA is substituted for by mevalonic acid, its salts, hydrates, and saponates, or the isoprenoid donors geranylgeraniol, geraniol, or farnesol.
